(19) United States
(12) Patent Application Publication
(10) Pub. No.: US 2006/0111944 A1
(43) Pub. Date: May 25, 2006

(54) System and method for encouraging performance of health-promoting measures

(76) Inventors: James R. Sirmans JR., Gainesville, FL (US); Kristofer F. Jurski, Fort McCoy, FL (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/293,702

(22) Filed: Dec. 2, 2005

Related U.S. Application Data
(63) Continuation-in-part of application No. 10/974,186, filed on Oct. 27, 2004.
(60) Provisional application No. 60/516,075, filed on Oct. 31, 2003.

(51) Int. Cl. A61B 5/00; G07G 1/14

(52) U.S. Cl. 705/3; 705/14

(57) ABSTRACT

The present invention relates to systems and methods for monitoring and promoting behavioral modification with regard to an individual's health. In particular, the present invention provides a means for monitoring an individual's performance of health-promoting measures and, based on the member compliance to health-promoting measures, issuing tangible and/or intangible incentive products. In one embodiment, exercise/activity monitors are worn by members to verify their identity and to record and transmit member compliance in performing health-promoting measures (i.e., heart rate, type of exercise, exercise intensity and duration). All health-related information, including physical examination results and recorded participation in health-promoting measures, are used to determine appropriate incentives (i.e., subsidize membership fees for health club) to be rewarded to said member.

### CROSS-REFERENCE TO A RELATED APPLICATION

This application is a continuation-in-part of pending U.S. patent application Ser. No. 10/974,186, filed Oct. 27, 2004, which claims the benefit of provisional patent application Ser. No. 60/516,075, filed Oct. 31, 2003, now abandoned, both of which are hereby incorporated by reference in their entirety.

### FIELD OF THE INVENTION

The present invention relates to a system and method for evaluating and monitoring member compliance with health-promoting measures and, depending on the level of member compliance, providing tangible and/or intangible incentive products.

### BACKGROUND OF THE INVENTION

Health care costs have risen steadily over the years. Controlling health care costs are mandatory for everyone, including businesses. For example, employers that contribute to employee insurance have seen dramatic increases in insurance premiums. Responsible managers in business need to develop systems to manage the optimal health of their employees, not only to save costs but also to save lives. Unfortunately, there is currently little that an employer can do to encourage employees to minimize medical expenses and/or to adopt a healthy lifestyle.

The traditional paradigm of insurance for health care services rendered is based on the current health care model-of treating symptoms of pain and/or dysfunction only after presentation in the patient. Thus, indemnification is provided after “pain/illness” occurs, which results in a costly, non-viable model for benefit delivery. Moreover, insured members often attempt to draw the maximum possible benefits (maximal use of health care service/providers) from their insurance packages due to a belief that contributions to the insurance package would otherwise be “wasted.” Insurers have failed to recognize that the number of “pain/illness” occurrences can be decreased if members were provided-incentives to perform health-promoting measures that would optimize their health/wellness (i.e., proper nutrition, exercise).

Studies have shown that costs related to excess medical utilization can be significantly reduced by proactively preventing detectable health risks, which are likely to become insurance claims. For example, “sustainable” exercise programs have been proven to dramatically improve an individual's health (morbidity) by reducing the incidence of sickness and/or injury, thus resulting in a longer life (improved mortality).

Moreover, the frequency of doctor visits or frequency of medication prescription varies statistically from persons who regularly exercise and make an effort to maintain their physical and mental health versus persons who do not do so. For example, individuals who regularly exercise and/or make an effort to maintain their health incur fewer medical expenses (i.e., fewer doctor visits, fewer medications) than an individual who does not exercise. Unfortunately, once insured, members may become apathetic in their efforts in maintaining a healthy lifestyle due to a view that any health related issues would be covered by insurance.

The existing insurance system ignores efforts by individuals to maintain their own health and wellness on a day-to-day basis. What is generally considered when a person applies to join an insurance plan are criteria such as the prospective member's age, sex, and data concerning the individual's past history. However, the insurance company does not take into account those “healthy” members who become apathetic in their efforts in maintaining their health as a result of a belief that any future health care costs would be covered by insurance.

Moreover, current insurance products lack control measures that assure member compliance with specified health measures for promoting health/wellness. Underwriting and pricing of insurance coverage is presently done at the time of application with eligibility for coverage based on data and representations from the member. Further, “lifestyle” issues such as smoking and alcohol consumption are taken at face value based on the member's representations. Members have little incentive to participate in health-promoting measures. Moreover, there are few measures available to verify the truthfulness of member representations or review member activities related to health/wellness on an on-going basis. Thus, a new insurance paradigm is needed to not only address caring for the sick but to also promote and reward fitness/wellness.

A system and method for monitoring individual efforts in maintaining health, which is reflected in their insurance premium, has been described in Japanese Patent No. 2002263071. The invention concerns insurance premiums that are discounted according to certain reported health-related activities performed by a customer, including utilization frequency by a customer of a sport club. However, recording the number of times a person attends a sports club does not accurately assess whether a person has performed any exercise activity, if at all. Moreover, it is not inconceivable that a member, in order to receive the insurance premium discount, would get another individual to impersonate him and create a spurious sports club attendance file.

Another method of monitoring health and discounting insurance premiums is provided in U.S. Patent Application Serial No. 2002/0013717. This invention concerns a portable, individualized exercise monitor that functions to assess user exercise activity as well as user identity. The monitor is designed to be connected to the body of the user, i.e., a pedometer, and includes a means for checking the identity of the person wearing the monitor. Unfortunately, this system can be very intrusive to an individual and be distracting during exercise activities. Moreover, the monitoring devices do not provide routine-specific identity (i.e., the specific exercise activity being performed, the specific muscle groups being impacted).

Other insurance savings schemes have been proposed in which members claim benefits at a lower rate than normal and/or can accrue funds in a savings account. These schemes, however, do not provide an incentive for members to maintain healthy lifestyles nor do they include a means for accurately monitoring an individual's compliance with requisite exercise/health activities for maintaining health and wellness.

### BRIEF SUMMARY OF THE INVENTION

The subject invention concerns novel systems and methods for encouraging an individual to perform health-promoting measures. In particular, the subject invention promotes a behavioral modification by providing tangible and intangible incentive products upon individual compliance in performing health-promoting measures. To do so, the subject invention further provides systems and methods for monitoring an individual's performance of such health-promoting measures.

In one embodiment, a system and method are provided for implementing and/or administering incentives to individuals who have subscribed to an organization that monitors (such as a gatekeeper) performance of health-promoting measures. A monitoring device (such as a personal monitoring device or monitoring system at a health-promoting location) is provided that observes and records the individual's (hereinafter also referred to as a “member”) efforts to perform health-promoting measures. The data from the monitoring device is received via a data communication channel and automatically mapped and incorporated into a member profile database. The profile database is used to issue tangible and/or intangible incentive products based on the member's performance history.

In accordance with the present invention, systems and methods for assessing member history (i.e., medical history & lifestyle) and/or member compliance in participating in health-promoting measures (i.e., exercise regimen) are provided. Incentives to participate in programs based on the subject invention include, but are not limited to, physical well-being, mental well-being, improved quality of life, monetary rewards, foods, gift certificates, marketing products, gift items, and the like. For example, incentives for insurance members to participate in insurance programs based on the subject invention can include financial incentives (i.e., reduction of insurance premiums based on member compliance with prescribed performance protocol regimen), and payment or reimbursement of costs related to the participation in activities designed to promote health/wellness (i.e., reimbursement for health club membership, reimbursement for weight loss program membership).

The systems and methods of the subject invention include a means for monitoring member performance of health-promoting measures. In one embodiment, an exercise machine is equipped with a means for monitoring member exercise activity. In a related embodiment, the exercise machine includes a means for monitoring individual identity; historical exercise activity; type of exercise, exercise intensity, and exercise duration; as well as physiological measurements (i.e., resting, activity, and recovery heart rates and pulse, blood pressure, temperature).

In another embodiment, a personal monitoring device is provided to a member, wherein the monitoring device is equipped with a means for continuous monitoring of member physiological status. Physiological data that can be monitored using a monitoring device of the subject invention include, but are not limited to, body core temperature, galvanic skin response, pulse, blood pressure, respiration, activity, heat flow, and certain electrical currents associated with electrocardiogram and electroencephalograph measurements. The personal monitoring device of the subject invention is preferably adapted to generate and provide feedback data to a database for recording the member's activity, including the degree to which a member has complied with a health promoting measure.

Based on the recorded participation in health-promoting measures (i.e., exercise activity) and other health information (i.e., improvement in medical condition/health), respective incentives (also referred to herein a rewards) are allocated to the member.

In accordance with the subject technology, a statistical individual profile data management continuum file is established for each member, in which the member's vital health statistics and prescribed exercise regimen are profiled, tracked, and stratified into a performance model based on protocol criteria.

In one embodiment, these protocol criteria are entitled, “Fitness Index Ratings.” The member is offered various levels of“performance” criteria, in which each level is associated with a specific incentive or points. Based on the chosen performance level, the member's compliance is monitored. For example, the member's historical attendance at a health club, along with the member's specific exercise regimen/performance data including, but not limited to, type of exercise performed; intensity level (calories burned), and duration, are documented, recorded, and communicated onto a computer-based system. The cumulative results from the member's performance data are then summarized into a legend. The legend provides a protocol compliant assessment summary that can be used to assess if and how much of a incentive or number of points will be issued to the member. This summary can be periodically transmitted to a third party (such as an insurer; a physician; state and federal agencies, etc.) for application toward related subject matter (such as coverage assessment; health assessment; statistical analysis; etc.).

In a preferred embodiment, the health-promoting measure can include specific exercise regimens to be performed in, or out of a health club. In a related embodiment, the individual wears a member monitoring device which uniquely identifies the individual, then gathers and relays data on member compliance and performance with regard to the chosen health promoting regimens (i.e., exercise regimens). The data from a personal monitoring device can then be relayed to a gatekeeper/database (such as an insurance provider) for monitoring and tracking member performance.

The invention can be implemented in numerous ways, including as a system (including a computer processing system, monitoring technology, and means for sensing member physiological status), a method (including a computerized method of statistically analyzing member physiological status data), an apparatus, a computer readable medium, a computer program product, or a data structure tangibly fixed in a computer readable memory. Several embodiments of the invention are discussed below.

As a COMPUTER SYSTEM, an embodiment of the invention includes a communications interface for receiving data input (i.e., from a sensor) regarding member activity, a database or other storage means, a display device and a processor unit. The display device has a plurality of display areas (windows). The processor unit operates to receive recognized data from a monitoring device and/or exercise machine communicated therefrom into a database (or other text based program) of the computer system.

As a METHOD of data entry and analysis, an embodiment of the invention includes the operations of: (a) receiving data regarding member activity via commonly available communication means (i.e., digital internet, etc.); (b) statistically analyzing the data to track and provide a profile of member activity; and (c) assessing the analyzed data to identify incentives.

As a COMPUTER READABLE MEDIA containing program instructions for data entry, an embodiment of the invention includes: computer readable code devices for (a) receiving member activity via communication means; (b) statistically analyzing the data to track and provide a profile of member activity; and (c) assessing the analyzed data to identify incentives.

The methods of the present invention may be implemented as a computer program product with a computer-readable medium having code thereon. The program product includes a program and a signal bearing media bearing the program.

Thus, the present invention provides the foundation on which an organization can justify providing incentives to encourage members to adopt and/or continue with health/wellness activities.

### BRIEF DESCRIPTION OF THE DRAWINGS

FIG. 1 is a flow chart illustrating the steps of evaluating an insurance product based on member performance of health-promoting measures according to the present invention.

FIG. 2 is a flow chart illustrating the steps of evaluating an incentive to be provided based on member performance of health-promoting measures according to the present invention.

### DETAILED DESCRIPTION OF THE INVENTION

The subject invention concerns systems and methods for encouraging an individual to perform health-promoting measures and to promote behavioral modification by providing incentives for compliance in performing the health-promoting measures. More specifically, this invention is an individual performance processing system that monitors individual performance of health-promoting measures and, based on individual performance, issues appropriate incentives/rewards.

In one embodiment, the subject technology provides a system and method for providing to insurance carriers verifiable data regarding member performance of health-promoting measures, on an on-going basis. Members qualify for financial incentives (such as discounted premiums and/or benefits) based on whether certain minimal acceptable standards of performance (also referred to herein as protocol criteria) in adopting and/or maintaining health-promoting measures are met, as prescribed by the insurance carriers. The financial incentives can be withdrawn should members fail to meet established protocol criteria (for example, a member falls short of prescribed pre-determined levels of performance). This, in turn, will make insurance coverage more cost-effective, more accessible to prospective members (lower cost), and will promote healthier members.

As used herein, the term “health-promoting measures,” refers to recordable and/or verifiable activities that are designed to improve and/or maintain individual health/wellness. Examples of health-promoting measures as contemplated herein include, and are not limited to, exercise regimens (i.e., weight training, aerobic exercises (i.e., swimming, kickboxing, jogging, bicycling, dancing, brisk walking, playing sports, etc.)); programs designed to improve wellness (i.e., yoga, smoke-ending courses, alcoholic-ending courses, weight control courses); wellness visits to the physician (i.e., annual breast exam; prostate examination); and preventive care (i.e., vaccinations, pap smears). Accordingly, member performance of health-promoting measures can include, but is not limited to, exercise; activities dedicated to improving or maintaining mental and emotional balance and health; activities dedicated to improving or maintaining relationship wellness; and activities dedicated to improving or maintaining nutritional or dietary habits.

The term “gatekeeper,” as used herein, refers to an organizational entity or computer system that continuously manages recorded data of monitored member performance of health-promoting measures. In a preferred embodiment, the gatekeeper profiles, tracks, and stratifies the recorded data into a performance model based on previously established protocol criteria regarding health-related measures. In one embodiment, the gatekeeper can assess the performance model and has the ability to issue incentives to individuals based on individual performance profiles.

An organizational entity, according to the present invention, can include any body that has a membership acting or united in a common purpose. For example, an organizational entity of the invention includes, but is not limited to, a corporation (such as an employer; insurance carrier; health club/spa; hospital; and the like) or individuals/members placed into an organizational entity for monitoring purposes or for establishing performance profiles. In a particular embodiment, the gatekeeper is an organizational entity that summarizes and provides the results of the performance model to a third party (such as an insurance company, physician, employer, federal agency, state agency, etc.), which can then determine member eligibility for rewards.

Specifically, the present invention provides systems and methods for assessing member health, which include but are not limited to, assessing the member's medical history, lifestyle, and member subscription to health-promoting measures (i.e., exercise regimen).

In certain embodiments, incentives to participate in such organizations (such as an insurance policy) include, but are not limited to, physical and mental well-being, improved quality of life; financial incentive features (such as a reduction in insurance premiums or physician office co-payments based on member compliance with a prescribed performance/protocol regimen or payment/reimbursement of costs related to member participation in such protocol (i.e., reimbursement for health club membership, payment for membership with Weight Watchers)); food (such as fruit baskets, etc.); merchandise and service prizes (such as DVD, camera, clothing, etc.); gift certificates, coupons, or cards (such as a gift certificate to a favorite store); and intangible property (such as stocks, bonds, mutual funds, etc.).

In accordance with the present invention, data provided by an exercise machine and/or monitoring device are transmitted to a central computer of the invention. A central computer of the invention, records and processes data provided by the monitoring device and/or exercise machine worn by the user. The central computer can be continuously accessed by a gatekeeper to profile, track, and stratify the data into a performance model based on pre-established criteria for health-promoting measures. Alternatively, an interested third party (such as an employer, an insurance carrier, a physician, a government agency, etc.) can access the central computer directly to determine member eligibility for incentives, provided the member has performed a minimum number of health-promoting measures.

The central computer, according to the subject invention, can be housed within a facility that is remotely located from the insurance carrier or can be housed with the insurance carrier. In a preferred embodiment, the central computer is housed within an insurance carrier facility while a patient performs health-promoting measures at a remotely located location from the insurance carrier.

Preferably, the central computer of the invention comprises a central processing unit (CPU) having sufficient processing power to perform program codes and algorithm operations in accordance with the subject invention. The program codes and algorithm operations, including the statistical data management continuum program and filtering, analysis, and monitoring operations, can be embodied in the form of computer processor usable media, such as floppy diskettes, CD-ROMS, zip drives, non-volatile memory, or any other computer-readable storage medium, wherein the computer program code is loaded into and executed by the central computer. Optionally, the program codes and/or operational algorithms of the subject invention can be programmed directly onto the CPU using any appropriate programming language, preferably using the C programming language. The central computer can also include a neural network for pattern recognition. Artificial Neural Networks ANNs are self learning; the more data presented, the more discriminating the instrument becomes. By running many standard samples and storing results in computer memory, the application of ANN enables the device to “understand” the significance of the monitored member performance of health-promoting measures better and to use this information for future analysis (for example, to analyze whether a member is getting healthier). “Learning” is achieved by varying the emphasis, or weight, that is placed on the output of one monitored health-promoting measure versus another. The learning process is based on the mathematical, or “Euclidean,” distance between data sets. Large Euclidean distances represent significant differences in sample-to-sample aroma characteristics.

In certain embodiments, the central computer comprises a memory capacity sufficiently large to perform program codes and/or algorithm operations in accordance with the subject invention. The memory capacity of the invention can support loading a computer program code via a computer-readable storage media, wherein the program contains the source code to perform the program codes and/or operational algorithms of the subject invention. Optionally, the memory capacity can support directly programming the CPU to perform the operational algorithms of the subject invention. A standard bus configuration can transmit data between the CPU, memory, ports and any communication devices.

In addition, as understood by the skilled artisan, the memory capacity of the central computer can be expanded with additional hardware and with saving data directly onto external mediums including, for example, without limitation, floppy diskettes, zip drives, non-volatile memory and CD-ROMs.

In certain instances, the central computer can also perform any one or combination of the following activities: (1) host/remote communications: the transfer of member records and status between the central/host system and remote systems for global update; (2) purge: the deletion of member records and status that are obsolete based on specified purge limits contained in the system control file; and (3) backup: the regularly invoked backup activity of member records and status files. Those skilled in the computer arts would readily understand how to implement such activities on a central computer.

The central computer can further include the necessary hardware and software to provide analyzed monitored information into an output form readily accessible by the insurance carrier, trained physician, technician, or insurance member. For example, without limitation, an audio device in conjunction with audio speakers can relay monitored and analysis results into an audio signal, and/or a graphical interface can display results in a graphical form on a monitor and/or printer. Further, the central computer can also include the necessary software and hardware to receive, route and transfer data to and from a remote location in which the portable device is in use.

More than one exercise terminal and/or monitoring device can communicate with the central computer of the invention at any time to form a network. The network can be arranged as a local area network (LAN), a wide area network (WAN), or a wireless network. Communications between terminals and/or monitoring devices and the central computer can be provided via wireless networks, coaxial cable, a twisted pair, optical fiber, or any other conventional communications link.

In one embodiment, the health-promoting measure to be monitored is the use of an exercise machine by an member. Any exercise machine that is commonly used to promote fitness is suitable for the present invention. Contemplated exercise machines include, by way of example, stationary bicycles, stair climbers, steppers, rowing machines, ski machines, treadmills, cross trainers, hiking machines, abdominal exercise machines, elliptical exercise machines, and strength-training and weight machines. According to the subject invention, such exercise machines include a terminal that communicates with a central computer of the invention.

A variety of highly sophisticated exercise machines that include terminals that can communicate to a central computer have been developed. For example, U.S. Pat. No. 6,447,424 describes hiking machines, treadmills, and the like, which include displays of mountainous terrain to provide visual enjoyment to the user while exercising. Such exercise machines can be used in accordance with the subject invention to communicate directly with a central computer or by synchronizing data to the member worn monitoring device.

An exercise machine preferably includes a means for recording the identity of the individual using the exercise machine and a means for monitoring an individual using the machine. The exercise machine preferably monitors the type of exercise being performed by the individual, the intensity and duration of the exercise activity, as well as physiological conditions such as heart rate or blood pressure, caloric energy expended, or number of steps taken. The means for monitoring an individual using an exercise machine and recording the identity of the individual can be based on known computerized devices and sensors, such as those disclosed in U.S. Pat. Nos. 6,458,060; 6,059,692; 6,053,844; 6,050,924; 6,033,344; 6,014,432; and 5,967,975.

In yet another embodiment, the health-promoting measure(s) to be monitored in accordance with the subject invention is member physical activity. Any personal monitoring device that can monitor member physiological status is suitable for the present invention. According to the subject invention, such personal monitoring devices include at least one sensor for the detection of physiological status data and a transmitter that is adapted to take data provided by the sensor(s) and transmitting the same to a central computer of the invention.

A monitoring device of the invention includes a means for recording the identity of the individual wearing the monitoring device and at least one sensor for monitoring the physiological status of the member wearing the monitoring device. The sensor(s) of the monitoring device can preferably monitor the type of exercise being performed by the individual, the intensity and duration of the exercise activity, as well as physiological conditions such as heart rate or blood pressure, caloric energy expended, or number of steps taken. The sensor(s) for monitoring physiological status data of an individual and the means for recording the identity of the individual can be based on known monitoring devices and sensors, such as those disclosed in U.S. Pat. Nos. 6,635,015; 6,605,038; 6,595,929; and 6,527,711 (collectively assigned to BodyMedia, Inc.).

In addition to monitoring exercise regimens, the present invention also provides methods for monitoring health-promoting measures that are not related to exercise. In one embodiment, member participation in activities dedicated to, without limitation, improving or maintaining mental and emotional balance and health (i.e., relaxation/stress-reducing activities); relationship wellness (i.e., marriage counseling); and healthy nutritional or dietary habits (i.e., Weight Watchers visits with nutritional specialist); can be monitored and used to assess incentives to be presented to an member.

In one embodiment, monitoring of non-exercise related health-promoting measures entails recordation of member attendance at meetings or facilities related to wellness. Recordation of member attendance can be carried out via mechanical, computerized, or manual means, or any combination thereof. For example, member attendance can be recorded by, but not limited to, a person who manually writes down the name and time of member attendance; a computerized system that includes an event indicator (i.e., an event indicator monitor that can determine whether the user has performed a prescribed event) and an identification means; or a mechanical device such as a punch ticket machine that will provide verification of member attendance. In any event, only individuals authorized to access and input responsible data will be allowed to vouch for performance.

Non-limiting examples of wellness related meetings or facilities include rehabilitation facilities, weight control facilities/meetings (i.e., Jenny Craig or Weight Watchers), and meditation courses/yoga facility. Data regarding the attendance duration and/or participated activity can be recorded and communicated to a central computer.

Further, the present invention provides methods for recording member participation in wellness visits with a clinician. In one embodiment, clinical and/or attendance data regarding wellness visits can be communicated to the central computer of the invention. Wellness visits to access preventive care services such as regular measurements of body lipids (cholesterol, high-density lipoprotein (HDL), low-density lipoprotein (LDL), triglycerides), glucose, blood pressure, heart rate, weight, and body fat; annual analysis of pap smears, blood, urine, and saliva; annual physical examination; breast and gynecological examination; prostate examination; and vaccinations (i.e., flu shots) can be recorded by the clinician and communicated to the central computer.

In accordance with the present invention, raw data transfer from a monitoring means to a gatekeeper or database for member profiling can be based upon events/activity (i.e., every time a physical activity is performed) or on a regular/periodic basis (i.e., daily, monthly, etc.). Raw data can transferred via any communication protocol in a computer system, including but not limited to HTTP, FTP, GOPHER, NEWS, NNTP, MAILTO, and the like.

Where the data from the personal monitoring device is provided to the gatekeeper, this data can be analyzed and evaluated as to efficacy of member performance prior to providing it to an interested party (such as an employer, insurer, and the like) in order to justify the awarding of incentives to the member. In a particular embodiment, the data is communicated directly to an interested third party to allow the third party to establish an appropriate reward to a member who has met minimal or acceptable standards of performance of health-promoting measures.

In one embodiment of the invention, points are issued for any health-promoting measures performed by a member. In addition to point accrual based on member health-performance, points can also be awarded to induce members to subscribe to the organization and/or a specific health-promoting measure. The gatekeeper collects the points relating to the member's performance and stores it in a computer system. A member may track the number of points accumulated in the computer system.

In certain embodiments, members can also access the number of points other members have accumulated. Rewards issued to a member may be based on a predetermined amount of points accumulated for performing health-promoting measures or based on a comparison of the member's performance against other members of the organization.

Communication devices such as wireless interfaces, cable modems, satellite links, microwave relays, and traditional telephonic modems can transfer monitored data from either an exercise machine, a personal monitoring device, an exercise facility, a gatekeeper, or a recordation means from a non-exercise meeting or facility to a central computer via a electronic communication (such as a network), oral communication (such as a telephone), in person communication, facsimile communication, or written communication. Networks available for electronic transmission of data include, but are not limited to, local area networks, intranets and the open Internet. A browser interface, for example, NETSCAPE NAVIGATOR or INTERNET EXPLORER, can be incorporated into communications software to view the transmitted data.

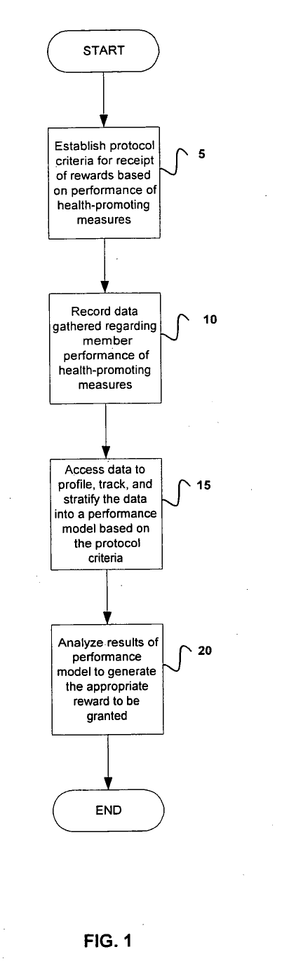

In accordance with the subject technology, as illustrated in FIG. 1, protocol criteria for receipt of incentives for member performance of health-promoting measures are established 5. Protocol criteria can be established by any entity that has appropriate knowledge or access to knowledge regarding human well-being and health including, but not limited to, an employer, an insurance carrier, a health professional, a governmental entity, self insured entity or any combination thereof.

Once the protocol criteria are established, they are presented to the member. In one embodiment, the member can select a protocol criteria level he or she wishes to perform from various levels of “performance” criteria. In another embodiment, the gatekeeper (i.e., insurance carrier) can select a protocol criteria level that a member must perform to receive any/all incentives offered.

Each member's recorded data in relation to activities associated with health-promoting measures 10 are stored in a central computer. A statistical data management continuum program of the central computer is initiated upon receipt of recorded member data. The statistical data management continuum program establishes a file for each member, in which the member's vital health statistics and prescribed exercise regimen are profiled, tracked, and stratified into a performance model based on protocol criteria 15. Each protocol criteria level is associated with a specific incentive (i.e., discounted premium payments). Based on protocol criteria level, either established or chosen, the member's compliance is monitored. For example, the member's historical attendance along with individual performance data including, but not limited to, type of exercise performed; intensity level, number of repetitions, are documented, recorded, and loaded onto a computer-based system.

In certain embodiments, all activity to be monitored (i.e., physical activity monitored by an exercise machine or a personal monitoring device of the invention) can be ‘benchmarked’ at the onset using a personal trainer to verify activities and the affect of same on caloric burn in a member.

In a related embodiment, where the physiological status data to be monitored by a personal monitoring device of the invention is the amount of calories burned, it is the ‘variance’ between resting/normal caloric burn and ‘activity-based’ caloric burn that will be recorded by the sensor(s) of the personal monitoring device. Such data can be used in accordance with the subject invention to provide evidence that the member is actively participating and complying with health promoting activities.

The cumulative results from the member's performance data are then summarized (i.e., “Protocol Compliant Assessment Summary”) to assess if and how much of a reward/incentive or points will be provided to the member 20. This summary can be generated by the central processing unit of a centralized computer or by a central processing unit of a gatekeeper, which can periodically transmit a continuous profile assessment. The continuous profile assessment can then be communicated to interested parties such as, but not limited to, the gatekeeper, the member, an insurer, etc. The continuous profile assessment can be used to assess whether and to what degree a reward is to be given to a member.

In one embodiment, protocol activity summaries will remain stored in the central computer and can be transferred to interested parties throughout the life of the member. This way, should a member move from one insurer to another insurer, the stored summaries can be accessed by the new carrier to assess an activity component which might affect premium rate determinations.

There are a variety of rewards (or incentives) available to encourage behavioral modification in members to participate in health-promoting measures. Suitable rewards include both tangible and intangible products/services.

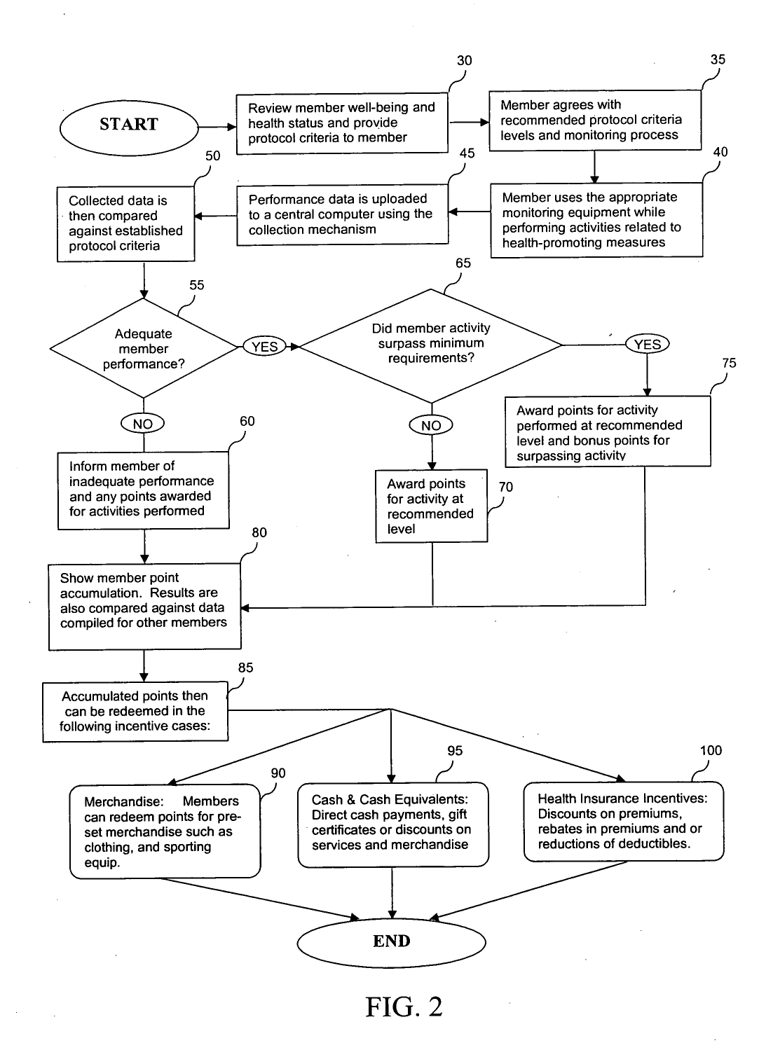

In accordance with the subject technology, as illustrated in FIG. 2, a member's well-being and health status are evaluated and, based on this evaluation, protocol criteria are established for recommended/minimum health-promoting measures to be performed as well as points to be awarded for member performance of health-promoting measures 30. In one embodiment of the invention, incentives to be awarded to members are stratified according to the number of points accumulated by a member.

Protocol criteria can be established by any entity that has appropriate knowledge or access to knowledge regarding human well-being and health including, but not limited to, an employer, an insurance carrier, a health professional, a governmental entity, self insured entity or any combination thereof.

Once the protocol criteria are established, they are presented to the member for member acceptance of both the protocol criteria as well as surveillance of member activity relating to health-promoting measures 35. For example, where the health-promoting measure is exercise and the surveillance constitutes monitoring equipment (such as a personal monitoring device or an exercise machine that includes a monitoring system), the member can agree to both the recommended/minimal level of exercise to be performed as well as appropriate use of the monitoring equipment.

In one embodiment, the member can select a protocol criteria level he or she wishes to perform from various levels of “performance” criteria. In another embodiment, the. gatekeeper (i.e., insurance carrier) can select a protocol criteria level that a member must perform to receive any/all incentives offered.

Each member's performance of activities associated with health-promoting measures is monitored 40, collected 45, and compared against the established protocol criteria 50.

In certain embodiments, as described herein, a statistical data management continuum program of the central computer is initiated upon receipt of recorded member data. The statistical data management continuum program establishes a file for each member, in which the member's vital health statistics and prescribed exercise regimen are profiled, tracked, and stratified into a performance model based on protocol criteria. For example, member performance in health-promoting activities is assessed to identify whether the established recommended/minimum level of performance was accomplished 55.

Should a member's performance not meet the established protocol criteria, the results regarding member inadequate performance and any points awarded for member activity will be reported to the member 60. Should the member's performance meet the established recommended/minimum level of performance, a further assessment on whether member's performance exceeds minimum requirements is then performed 65. Should the member's performance only meet the established protocol criteria, the points established in the protocol criteria are awarded to the member 70. Should the member's performance exceed minimum requirements, the points established in the protocol criteria as well as bonus points are awarded to the member 75.

The cumulative results from the member's performance data (including total points awarded) are then summarized (i.e., “Protocol Compliant Assessment Summary”) and provided to the member 80. In certain embodiments, the summary is compared against other member summaries to assess overall member population compliance in performing health-promoting measures.

The member performance summary can be generated by the central processing unit of a centralized computer or by a central processing unit of a gatekeeper, which can periodically transmit a continuous profile assessment. The continuous profile assessment can then be communicated to interested parties such as, but not limited to, the gatekeeper, the member, an insurer, etc. The continuous profile assessment can be used to assess whether and to what degree a reward is to be given to a member.

A member can redeem his points 85 to obtain a variety of incentive products such as: merchandise 90; cash/cash equivalents 100; or health insurance incentives 105.

According to the subject invention, any party can select the protocol criteria and/or types of incentive products to be presented to a member at any time during the process. For example, the gatekeeper, insurer, or employer can select which category of incentive product a member is to-receive at the onset or prior to member performance of any health-promoting activities. In other embodiments, the member can select which incentive product he would like to receive at any time during the process. For example, a member can select the incentive product he would like to receive at the onset and the performance summary tracks how close the member is to receiving the selected incentive product.

In one embodiment, where the individual is an insurance member of an insurance program, the reward is a decrease in premium payments, subsidized payment of fees associated with performing health-promoting measures (i.e., membership fees to a sports club, or subsidized payment of the exercise machine); and/or increased benefits payment according to a predetermined scheme.

In another embodiment, where the individual is a member of a program that promotes improved health, the incentive can be any one or combination of the following: monetary rewards (e.g., cash); gift cards, coupons, or certificates (e.g., gift certificate to a favorite store, amusement park, restaurant, etc.); financial incentives (e.g., stocks, bonds, funds, etc.); and merchandise and service prizes (e.g., MP3 player, DVD, cosmetic makeover, trip to exotic locations, and the like).

The present system can automatically generate a number of instructional materials for a member based on the member profile to address performance gaps of the member. Appropriate instructional material commensurate with health status and performance attributes of a member can be automatically created. Anyone, including a member, gatekeeper, or third party can view a list of available instructional material automatically generated for specific health-promoting measures. In certain embodiments, the gatekeeper can select the instructional material to be assigned to a member. Where the gatekeeper is a computer system, computer assigned instructional material can be generated using a computer adaptive instructional process that relies on retrieved member performance, health status and attributes, and monitored profile data. The member attributes or profile data can be retrieved from a database and include various member related information, such as gender, health status (i.e., whether the member has high blood pressure or cholesterol, age of the member, clinical history, injuries, etc.) as well as performance history (i.e., whether the member has continued to perform health-promoting measures).

Existing instructional materials can be customized for each member based on his or her profile data stored in the system of the present invention. For example, based on a clinical history identification parameter (which can, for example, be about the patient's history of high blood pressure) stored in a member profile database, the present invention can present instructional health-promoting measure material for a particular member, e.g. suggestion to eat less salt. Using one or more of access, interface and integration application layers, the system of the present invention can access other member information databases, e.g., public and private health system databases, to determine/retrieve member profile data and to transform the existing instructional material commensurate with the instructional needs of the member.

Based on the foregoing specification, the invention may be implemented using computer programming or engineering techniques including computer software, firmware, hardware or any combination or subset thereof. Any such resulting program, having computer-readable code means, may be embodied or provided within one or more computer-readable media, thereby making a computer program product, i.e., an article of manufacture, according to the invention. The computer readable media may be, for instance, a fixed (hard) drive, diskette, optical disk, magnetic tape, semiconductor memory such as read-only memory (ROM), etc., or any transmitting/receiving medium such as the Internet or other communication network or link. The article of manufacture containing the computer code may be made and/or used by executing the code directly from one medium, by copying the code from one medium to another medium, or by transmitting the code over a network.

One skilled in the art of computer science will easily be able to combine the software created as described with appropriate general purpose or special purpose computer hardware to create a computer system or computer sub-system embodying the method of the invention. An apparatus for making, using or selling the invention may be one or more processing systems including, but not limited to, a central processing unit (CPU), memory, storage devices, communication links and devices, servers, I/O devices, or any sub-components of one or more processing systems, including software, firmware, hardware or any combination or subset thereof, which embody the invention. User input may be received from the keyboard, mouse, pen, voice, touch screen, or any other means by which a human can input data into a computer, including through other programs such as application programs.

Following is an example that illustrates an embodiment of the invention. This example should not be construed as limiting.

### EXAMPLE 1

In one embodiment, a member will pay a monthly fee to an organization to receive a personal body monitor and access to a website portal. Wearing the body monitor, the member will be asked to perform the Center for Disease Control recommended levels of exercise (i.e., moderate intensity for 30 minutes a day 5 days a week and/or vigorous intensity for 20 minutes a day 3 days a week). Members can then upload their compliance data from their monitors over the Internet to the organization (which includes a computer system and member profile database). In certain instances, data from the personal body monitoring system is automatically relayed to the organization. In certain embodiments, the transferred data regarding member performance can be displayed to the member on a website.

In one embodiment, a point system is assigned to performance data. The organization can calculate how many points a member has earned for each day of exercise. For example, if a member was 100% compliant for the day, the member would receive 100 points. Accordingly, the more active a member is, the more points a member can in turn accumulate. There are also bonus points that can be assigned for various activities (such as bonus points for vigorous exercise; or if a member is compliant in performing health-promoting measures for the week, points are doubled).

In another embodiment, members are encouraged to compete with other members in virtual games, where points in the games correspond to member performance/points. In a related embodiment, contests/prizes for the member who burns the most calories over their resting metabolic rate are provided to members of the organization.

In another embodiment, a virtual race will be provided to members of an organization, wherein performance data from the personal monitor regarding distances traveled (or steps physically taken) by the member are applied to the virtual race. The virtual race will allow groups of members and individual members to race across a geographical location. For example, using the step information collected from the personal monitoring device, the information is translated to a distance traveled on a virtual map; thus, a member could virtually race from Miami to New York. According to the subject invention, the first, second and third place winners would either receive points and/or merchandise.

According to the subject invention, members can redeem their points for incentive products (such as merchandise or service prizes), where the value of the merchandise/service varies depending on the number of points redeemed. For example at the low end of the spectrum (where a relatively low number of points is redeemed), a member could receive DVDs or gift certificates to movie theaters. On the high end (where a relatively high number of points is redeemed), members could receive electronics such as I-pods, DVD players and radios as well as other merchandise such as trail bikes and exercise equipment.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

We claim:

1. A method for providing incentives to a member to perform health-promoting measures comprising:
a) establishing a list of at least one health-promoting measure and at least one incentive to be presented to the member based on member performance of at least one health-promoting measure;
b) monitoring the member's compliance in performing said at least one health-promoting measure; and
c) assessing the appropriate at least one incentive to be issued to the member based on monitored compliance.

2. The method according to claim 1, further comprising the step of using at least one incentive to the member based on the assessment.

3. The method according to claim 1, wherein a personal monitoring device that comprises at least one sensor and a transmitter is used to monitor the member's compliance in performing the health-promoting measure.

4. The method according to claim 1, wherein the at least one health-promoting measure is any one or combination of the group of activities selected from the group consisting of exercise performed; activities dedicated to improving or maintaining mental and emotional balance and health; activities dedicated to improving or maintaining relationship wellness; and activities dedicated to improving or maintaining nutritional or dietary habits.

5. The method according to claim 4, wherein the exercise is any one or combination selected from the group consisting of: activities on an exercise machine, aerobic activities, and weight training; wherein the exercise machine is selected from the group consisting of stationary bicycles, stair climbers, steppers, rowing machines, ski machines, treadmills, cross trainers, hiking machines, abdominal exercise machines, elliptical exercise machines, and strength-training and weight machines.

6. The method according to claim 4, wherein the activity directed to improving or maintaining mental and emotional balance and health comprises attending meditation courses or yoga classes.

7. The method according to claim 6, wherein the health-promoting measure is the activity directed to improving or maintaining mental and emotional balance and health, wherein the step of monitoring the member's compliance in improving or maintaining mental and emotional balance and health comprises recording member attendance.

8. The method according to claim 4, wherein the activity directed to improving or maintaining nutritional dietary habits comprises meetings to address weight control meetings, or visits with a nutritional specialist.

9. The method according to claim 1, wherein the health-promoting measures are wellness visits with a clinician.

10. The method according to claim 1, wherein the incentive is any one or group selected from the group consisting of: monetary rewards; gift cards, coupons, or certificates; financial incentives; and merchandise and service prizes.

11. A system for encouraging member performance of health-promoting measures, said system comprising:
a) a means for monitoring a member's performance of at least one health-promoting measure;
b) a centralized computer;
c) a means for communicating monitored performance of at least one health-promoting measure to the centralized computer; and
d) a means for assessing appropriate incentives to be issued to the member based on monitored performance.

12. The system according to claim 11, wherein the means for monitoring the member's performance is a personal monitoring device that comprises at least one sensor and a transmitter.

13. The system according to claim 11, wherein the centralized computer comprises a central processing unit.

14. The system according to claim 13, wherein the central processing unit performs program codes for a statistical data management continuum program.

15. The system according to claim 13, wherein the central processing unit performs program codes to summarize the member's performance of the at least one health-promoting measure.

16. The system according to claim 14, wherein the at least one health-promoting measure is a non-exercise related health-promoting measure and the means for monitoring comprises a recordation means.

17. The system according to claim 16, wherein the recordation means is selected from the group consisting of a person, a computerized system, or a mechanical device.

18. The system according to claim 11, wherein the incentive is any one or group selected from the group consisting of: monetary rewards; gift cards, coupons, or certificates; financial incentives; and merchandise and service prizes.

19. A computer readable medium containing program instructions for displaying member data on a display device of a computer system, the data being obtained from tables in a database associated with the computer system, said computer readable medium comprising:
first computer program code for receiving data regarding a member's health;
second computer program code for establishing a list of at least one health-promoting measure and at least one incentive to be presented to the member based on the assessment of health;
third computer program code for assessing the member's health;
fourth computer program code for monitoring the member's compliance in performing said at least one health-promoting measure based on assessment of health; and
fifth computer program code assessing the appropriate at least one incentive to be presented to the member based on monitored compliance.

20. A computer readable medium containing program instructions for displaying member data on a display device of a computer system, the data being obtained from tables in a database associated with the computer system, said computer readable medium comprising:
first computer program code for establishing a list of at least one health-promoting measure;
a second computer program code for monitoring the member's performance of said at least one health-promoting measure based on data communicated from a monitoring means;
a third computer code for comparing the member's performance of said at least one health-promoting measure with other member performances; and
a fourth computer program code for assessing an appropriate at least one incentive to be presented to the member based on the comparison of performances.

21. The computer readable medium program of claim 20, wherein the display is provided as a virtual game.
